(19) United States
(12) Patent Application Publication
(10) Pub. No.: US 2023/0301813 A1
(43) Pub. Date: Sep. 28, 2023

(54) ANKLE FOOT ORTHOSES

(71) Applicant: Joseph B. SIA, Clifton, NJ (US)

(72) Inventor: Joseph B. SIA, Clifton, NJ (US)

(21) Appl. No.: 18/082,124

(22) Filed: Dec. 15, 2022

Related U.S. Application Data
(60) Provisional application No. 63/324,206, filed on Mar. 28, 2022.

(51) Int. Cl. A61F 5/01

(52) U.S. Cl. CPC A61F 5/0113; A61F 2005/0165

(57) ABSTRACT

An ankle foot orthosis (AFO) for use on the lower limb of a person involves a shell having a phalangeal section, a heel section, and an upright portion coupled to the heel section dimensioned to extend, substantially perpendicular to the heel section, from the heel section, and wherein at least the phalangeal section is flexible in a plantar flexation direction; and wherein the upright portion is substantially rigid so as to inhibit plantar flexion of the person's foot.

### FIELD OF THE INVENTION

This disclosure relates generally to biomechanical devices and, more particularly, to devices for a lower limb affected by a drop foot condition.

### BACKGROUND

Foot drop is a common disability, affecting millions worldwide. People suffering from foot drop struggle with limited mobility and are at increased risk of injury from slips and falls. While foot drop usually only affects one foot, in some cases both feet may be affected. In addition, depending on the cause, foot drop can be a temporary or permanent condition.

Foot drop is a muscular weakness that makes it difficult to lift the front part of the foot and toes and can cause involuntary plantar flexion when walking.

Foot drop is the result of weakness or paralysis of the muscles that lift the front part of your foot, often, but not exclusively, caused by compression of the nerve that controls the muscles that lift the foot, nerve damage in the leg, or as a result of a brain or spinal injury. Inherited conditions that cause peripheral nerve damage and muscle weakness, such as Charcot-Marie-Tooth disease (hereditary motor and sensory neuropathy), may also lead to foot drop.

Irrespective of the underlying cause, patients suffering from foot drop tend to experience similar difficulties such as difficulty lifting, turning, or flexing the affected foot. Consequently, without some form of support to control the foot's position, tripping over objects, miss-stepping, or falling can result.

In order to stabilize the foot of a person with a drop foot condition, an ankle foot orthosis (“AFO”) is often used. The AFO is a type of brace that supports the weak muscles and thereby constrains movement of the sole of the affected foot beyond about a 90 degree angle relative to the bones of the lower leg.

Common AFOs present problems. They are often large, bulky and/or heavy and uncomfortable. They can impinge on the ankle, causing discomfort, immediately or over time. Depending upon their particular configuration, they can also adversely affect blood flow through the dorsum. Some AFOs are also difficult to put on, particularly if the person has limited mobility, thereby often requiring the assistance of another person. Still further, common AFOs are designed such that they must be put on the affected foot prior to inserting the foot into the person's footwear, making that act difficult as well. Finally, because of their shape or bulk, many AFOs limit the type of footwear the user can wear.

Thus, there is a continuing need for AFO designs that can address one or more of the foregoing problems.

### SUMMARY

I have devised a solution that provides a significant advance in addressing the aforementioned problem.

One aspect involves an ankle foot orthosis (AFO) for use on the lower limb of a person, the AFO having a thermoplastic shell involving a phalangeal section dimensioned to underlay the person's phalanges bones, a heel section dimensioned to at least underlay the person's cuneiform bones and calcaneus, and an upright portion coupled to a proximal portion of the heel section dimensioned to extend, substantially perpendicular to the heel section, from the heel section to just below the gastrocnemius muscle, while covering both the calcaneal tendon and at least a portion of the posterior soleus, while not covering the ankle joint. At least the phalangeal section is flexible in a plantar flexation direction and the upright portion is substantially rigid so as to inhibit plantar flexion of the person's foot.

Another aspect involves an ankle foot orthosis (AFO) for use on the lower limb of a person, the AFO having a polycaprolactone shell, including a phalangeal section dimensioned to underlay the person's phalanges bones, a heel section dimensioned to at least underlay the person's cuneiform bones and calcaneus, and an upright portion coupled to a proximal portion of the heel section dimensioned to extend, substantially perpendicular to the heel section, from the heel section to just below the gastrocnemius muscle, while covering both the calcaneal tendon and at least a portion of the posterior soleus, while not covering the ankle joint. At least the phalangeal section is flexible in a plantar flexation direction and the upright portion is substantially rigid so as to inhibit plantar flexion of the person's foot.

Still another aspect involves a 3D printed ankle foot orthosis (AFO) for use on the lower limb of a person, the AFO having a shell including multiple layers of plastic, the shell further having a phalangeal section of dimensioned to underlay the person's phalanges bones, a heel section dimensioned to at least underlay the person's cuneiform bones and calcaneus, and an upright portion coupled to a proximal portion of the heel section dimensioned to extend, substantially perpendicular to the heel section, from the heel section to just below the gastrocnemius muscle, while covering both the calcaneal tendon and at least a portion of the posterior soleus, while not covering the ankle joint. At least the phalangeal section is flexible in a plantar flexation direction and the upright portion is substantially rigid so as to inhibit plantar flexion of the person's foot.

### BRIEF DESCRIPTION OF THE DRAWINGS

This disclosure is further described in the detailed description that follows, with reference to the drawings, wherein the same reference numbers appearing in the various drawings and description designate corresponding or like elements among the different views. and in which:

FIG. 1 illustrates, in simplified form, a perspective view of one example of an AFO 100 for use by a person and constructed according to the teachings herein;

FIG. 2 is a photograph of an example AFO constructed in a configuration as shown for the AFO of FIG. 1 following insertion into an article of footwear;

FIG. 3 illustrates, in simplified form, relevant dimensions H1, H2 of an example user's leg when standing, with the distance H1 measured between the center of the user's kneecap and the ground and the distance H2 measured between the same center of the kneecap and the top of the user's in step;

FIGS. 4-5 show various dimensions of the example AFO of FIG. 2;

FIG. 6 is a photograph of an example AFO that is similar to the AFO of FIG. 2, except that it lacks the ridge of FIG. 2 and includes two straps;

FIG. 7 illustrates, in simplified form, an alternative example AFO 700 according to the teachings herein;

FIG. 8A illustrates, in simplified form, an underside of a phalangeal portion having alternating thicknesses T1 and T2;

FIG. 8B illustrates, in simplified form, a side view of the underside 802 of FIG. 8A; and

FIG. 9 is a photograph of an example AFO 900 that is essentially an implementation of the AFO 700 of FIG. 7.

### DETAILED DESCRIPTION

I have developed AFO designs that, depending upon the particular implementation provide several advantages, including two or more of the following advantages. They are easy to wear, can be worn with socks and/or most any footwear, they do not cause pain after wearing for long periods of time, they do not interfere with the ankle or cause/aggravate edema of the ankle, they support the ankle and keep the foot dorsiflexed properly, they prevent the ankle and foot from inverting downwards or dropping, they do not interfere with blood circulation, and allow compensation for sensation and numbness due to nerve changes.

Some AFO design implementations constructed based upon the teachings herein can include flexible straps that allow the wearer to exercise the weakened muscles.

Other AFO design implementations constructed based upon the teachings herein can include multiple straps to provide different tension levels to increase ankle stability and/or provide greater or lesser resistance for purposes of exercise.

As used herein, the term “substantially” or “about” with respect to size, shape or thicknesses is intended to mean the value that is specified but subject to minor variations or local distortions in, as applicable, the size, shape or thickness, etc. that could occur in, for example, normal manufacture while being able to accomplish what is intended.

As used herein “substantially perpendicularly” is intended to mean, although deviating from the perpendicular (i.e., 90 degrees), sufficiently perpendicular such that the overall effect on the user in the context of an AFO, is the same as would be the case if the identified parts were precisely perpendicular to each other, for example, deviations from perpendicular of less than 3 to 5 degrees are intended to be “substantially perpendicularly.”

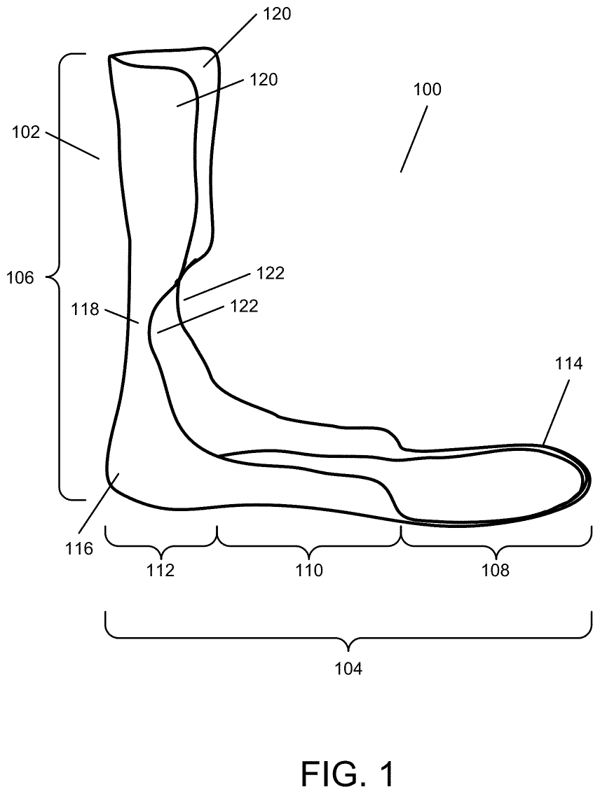

With the foregoing in mind, FIG. 1 illustrates, in simplified form, one example of an AFO 100 for use by a person and constructed according to the teachings herein.

As shown, the AFO 100 is made up of a shell 102 having a sole portion 104 and an upright portion 106, with the sole portion 104 and upright portion 106 being substantially perpendicular to each other.

The sole portion 104 is functionally comprised of three sections, a phalangeal section 108 dimensioned to underlay at least a person's phalanges bones, a metatarsal section 110 dimensioned to underlay at least a portion of a person's metatarsal bones, and a heel section 112 dimensioned to underlay a person's cuneiform and calcaneus bones.

The periphery of the phalangeal section 108 forms a ridge 114 that is high enough to maintain the phalangeal section 108 in a rigid position with respect to gravity forces when held oriented as it would be worn while standing (i.e., it prevents that section from flopping down), but otherwise allows the phalangeal section 108 of the sole portion to flex (e.g., when inserted into a user's footwear or during the stance phase of walking).

The heel section 112 includes a substantial heel wall 116 that couples the sole portion 104 to the upright portion 106 and forms a heel cup that inhibits plantar flexion of the person's foot during the swing phase of walking.

Depending upon the particular implementation, the metatarsal section 110 can include either a continuation of the ridge 114 of the phalangeal section 108 that allows for plantar flexure of the metatarsal section 110 or a continuation of the heel wall 116 that inhibits plantar flexion of the metatarsal section 110. As shown in the AFO 100 of FIG. 1, the latter is the case.

The upright portion 106 of the shell 102 will typically, and as shown, will be constructed to extend from the heel section 112 to, when on a person who is the user, a point typically just below the location of the user's gastrocnemius muscle, while a segment 118 covers and partially wraps around the part of the user's leg containing the user's calcaneal tendon and another segment 120 covers and partially wraps around, at least a portion of the user's posterior soleus. In addition, the segment 118 of the upright portion is narrower than the segment 120 of the upright portion so as to form a gap 122 that leaves the user's ankle uncovered.

In general, the upright portion 106 of the shell 102 is substantially rigid in a direction that serves to help prevent plantar flexion. It does this based upon the shape and by causing the shell to abut the user's leg during the swing phase when walking thereby preventing undesired plantar flexion during that phase of walking and, potentially, while the user is sitting in certain positions.

The phalangeal section 108 has a width sufficient to inhibit lateral movement of the AFO 100 and movement of the AFO towards the toe box of a user's footwear when the AFO 100 is fully inserted into a user's footwear. In this way, the user can insert the AFO 100 into their footwear and then insert their foot into that AFO 100 footwear combination.

Depending upon the particular implementation, the phalangeal section 108 of AFOs as described herein can be shaped specifically for a right foot, a left foot, or can be fairly generic or symmetrical shaped so as to be usable with either foot.

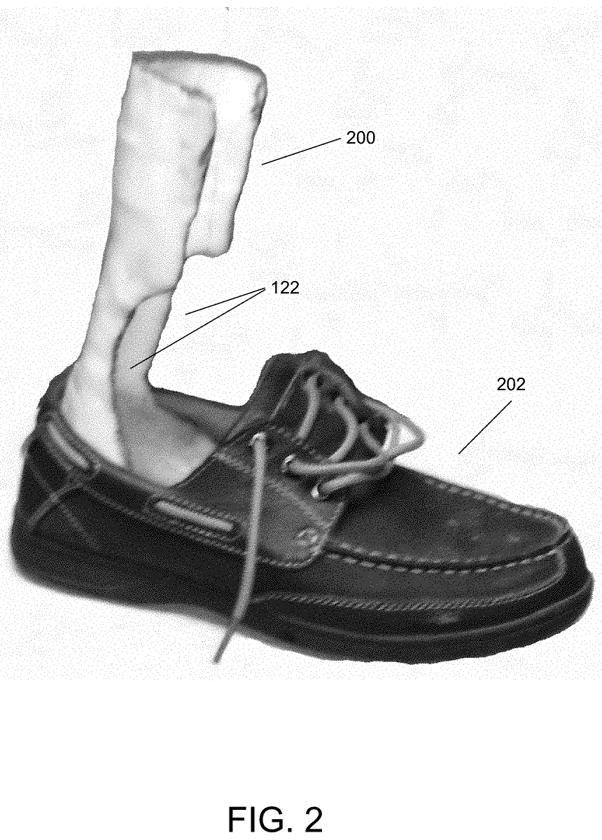

FIG. 2 is a photograph of an example AFO 200 constructed in a configuration as shown for the AFO 100 of FIG. 1 following insertion into an article of footwear 202.

As can be seen in FIG. 2, the gap 122 leaves clearance for the user's ankle so that irritation of the user's ankle does not occur.

For purposes of understanding, representative examples of dimensions of the AFO 200 of FIG. 2 will now be provided for an example male user who wears a size US7.5 shoe.

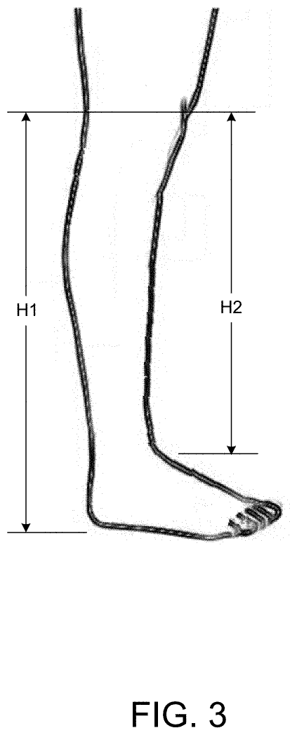

In conjunction with FIG. 2 and FIGS. 4-5, for purposes of understanding proportions, FIG. 3 illustrates, in simplified form, relevant dimensions H1, H2 of an example user's leg when standing, with the distance H1 measured between the center of the user's kneecap 302 and the ground 304 being 45.7 cm (18 inches) and the distance H2 measured between the same center of the kneecap 302 and the top of the user's in step being 39.5 cm (15.5 inches).

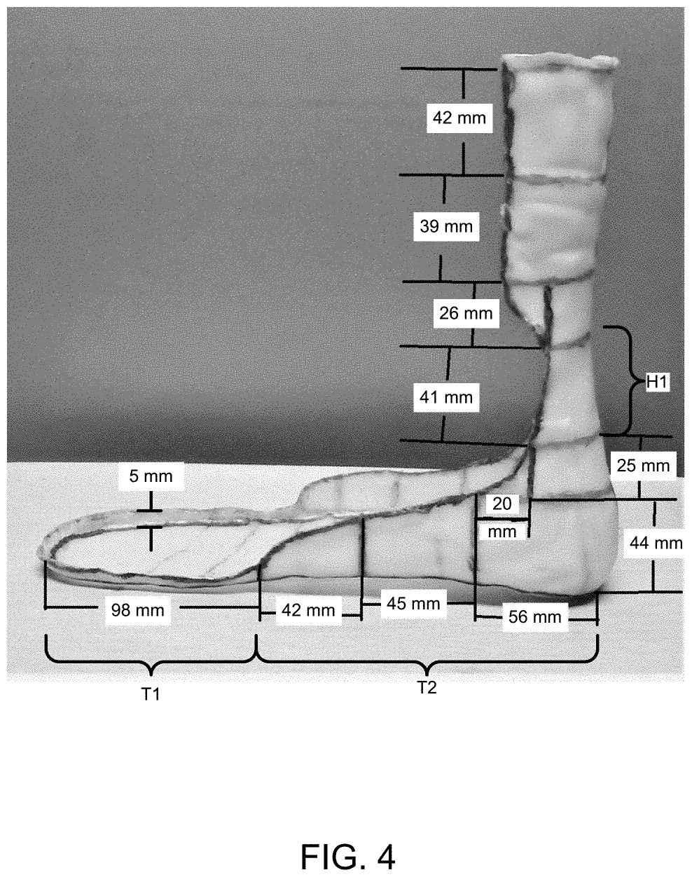

FIGS. 4-5 show various dimensions of the example AFO 200 of FIG. 2, bearing in mind that the lines shown thereon are not intended to be part of the AFO 200; they were drawn on the AFO 200 to merely show where the individual dimension was taken. In addition, as indicated in FIG. 4, the phalangeal section 108 has a thickness T1, in this example, substantially in the range of 1 mm to 1.5 mm, whereas the remainder of the sole portion 104 has a thickness T2 of substantially 2 mm. In general, T2 will be greater than T1 and, as noted above, the thickness T1 of the phalangeal section 108 should be sufficient to allow it to easily flex for insertion into footwear, but rigid enough that the ridge 114 will keep it from deflecting or “flopping” on its own. Moreover, in general, the thickness T2 should be selected so as, in conjunction with the heel wall 116, to substantially inhibit flexing.

The upright portion 106 has a thickness of between about 1 mm and about 1.5 mm, and typically about 1.5 mm, whereas the rest of the upright portion has a thickness of about 2 mm. This differential in thickness allows for easier insertion of the user's foot once the AFO 200 has been inserted into the footwear. It also allows for some dorsiflexion of the foot.

Optionally, if greater stability is required, or the ridge 114 cannot provide sufficient support against undesired plantar flexation alone, some implementations can include at least one strap, extending form the phalangeal section 108 to the segment 120. Note that the at least one strap can have at least a section that is elastically deformable, if desired, to allow for some nominal plantar flexion.

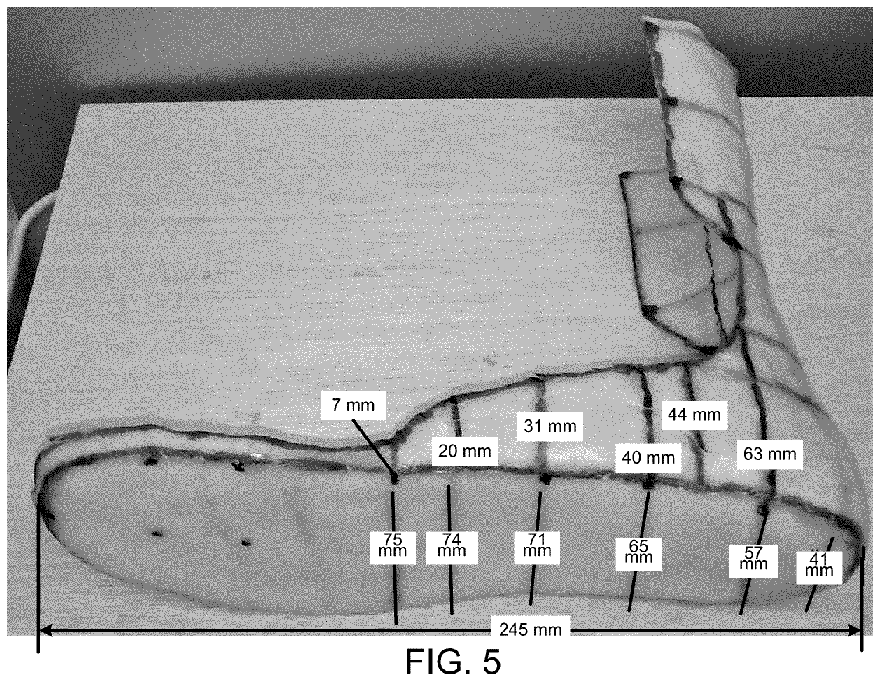

FIG. 6 is a photograph of an example AFO 600 that is similar to the AFO 200 of FIG. 2, except that it lacks the ridge 114 of FIG. 2 and includes two straps 602.

As shown in FIG. 6, the straps 602 are rigidly affixed to the phalangeal section 108, for example, by stitching, glue or as part of the shell formation process. Thus, the straps 602 are thin enough so that they do not cause foot discomfort. The opposite end of the straps includes a means of releaseably affixing the strap to the uppermost segment 120. Depending upon the particular implementation, this can involve, for example, one or more hook and loop fasteners (e.g., Velcro®), a hook that couples to an eyelet attached to, or hole in, the uppermost segment 120, a buckle to which the strap(s) 602 can attach, one or more snaps, or any other desired approach, the important aspect being the ability to releasably attach, not the means by which it is accomplished.

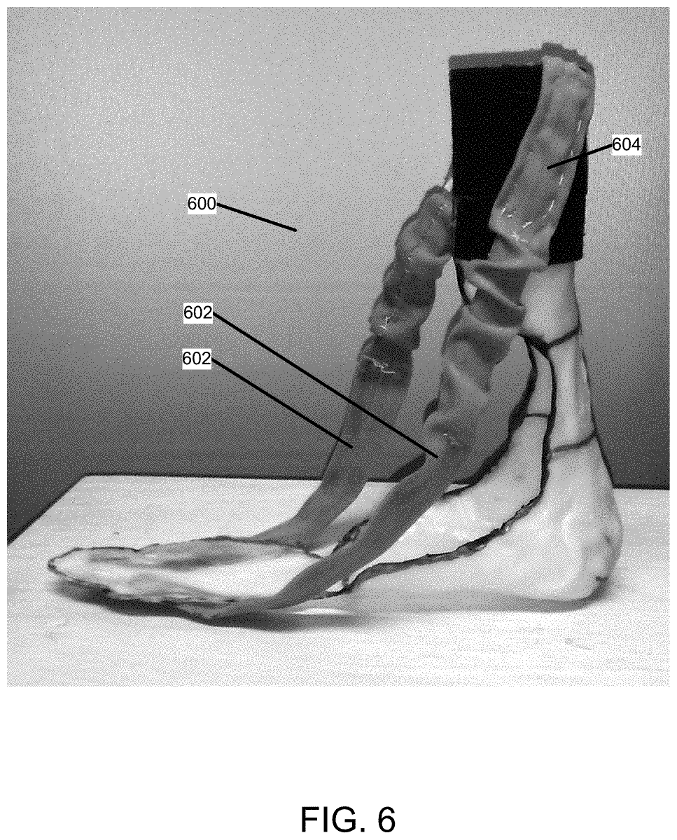

FIG. 7 illustrates, in simplified form, an alternative example AFO 700 according to the teachings herein.

As shown in FIG. 7, the AFO 700 is similar to the AFO 100 of FIG. 1, except that there is no ridge 114, the heel wall 116 is smaller and the section 118 of the upright portion 106 is narrower. As will be described below, the section 118 is sufficiently narrow as to allow some flexing in that area, for example, if the wearer stands on their toes or performs certain exercises involving plantar flexion. In addition, both the phalangeal section 108 and the metatarsal section 110 are between about 1 mm and 1.5 mm thick, whereas the heel section 112 and heel wall 116 is 2 mm thick. Thus, is a result of the lack of the ridge for both the phalangeal section 108 and the metatarsal section 110, the sole portion 104 is much more flexible. Again, the flexibility is to allow the AFO 700 to be inserted into a user's footwear before the user's foot is inserted therein and, advantageously, to allow for exercise through plantar flexion.

Finally, the AFO of FIG. 7 includes a pair of extensions 714 or “wings” positioned so as to be located within the quarter of a typical shoe just below the topline of the quarter of the shoe. The extensions 714 prevent side-to-side wobbling of the AFO 700 when in a shoe.

In addition, with the AFO 700 of FIG. 7, for purposes of walking, to counteract the flexibility of the phalangeal section 108 and the metatarsal section 110, at least one strap 702, and typically two, are present. The strap(s) 702 have two ends 704, 706, one end 704 of which affixed to the phalangeal section 108. The strap 702 is either entirely elastic or has one or more non-elastic sections 708 and an elastic section 710 somewhere within the length of the strap 702.

At or near the other end 706 of the strap(s) 702 is one part 712a of a connection mechanism, with the other part 712b being located on the uppermost segment 120. Depending upon the particular implementation, the connection mechanism 712a, 712b may be made up of the parts of a hook and loop fastener (e.g., Velcro®), a hook that connects to one of a series of holes or eyelets, a hook that can connect to one of a series of rings, a buckle, through which part of the section 708 near the end 706 of the strap 702 can be passed, a set of snap fasteners or anoy other type of fastener, the important aspect being the ability to release-ably fasten, not the mechanism used to do so. Alternatively, the strap 702 can be two parts, with one part being affixed to the uppermost segment 120 and the other part being affixed to the phalangeal section 108 and they release-ably connect to each other somewhere in between. Thus, when the strap 702 continuously attaches the uppermost segment 120 to the phalangeal section 108, platar flexion can be constrained or, resistance-based exercise of the muscles involved in plantar flexion can occur.

At this point it is worth noting that, an alternative, but less desirable configuration can achieve sufficient flexibility in at least the phalageal section 108 by having a series of alternating thicknesses of, for example, thickness T1 of between 0.8 mm and 1 mm, followed by T2 of >1.5 mm, followed by a thickness T1, followed by a thickness T2, etc. extending in a direction perpendicular to the longitudinal direction of the sole portion 104, with a “width” in the longitudinal section of a few mm or less, the width being a matter of design choice, provided the relevant flex of the phalangheal section 108 exists.

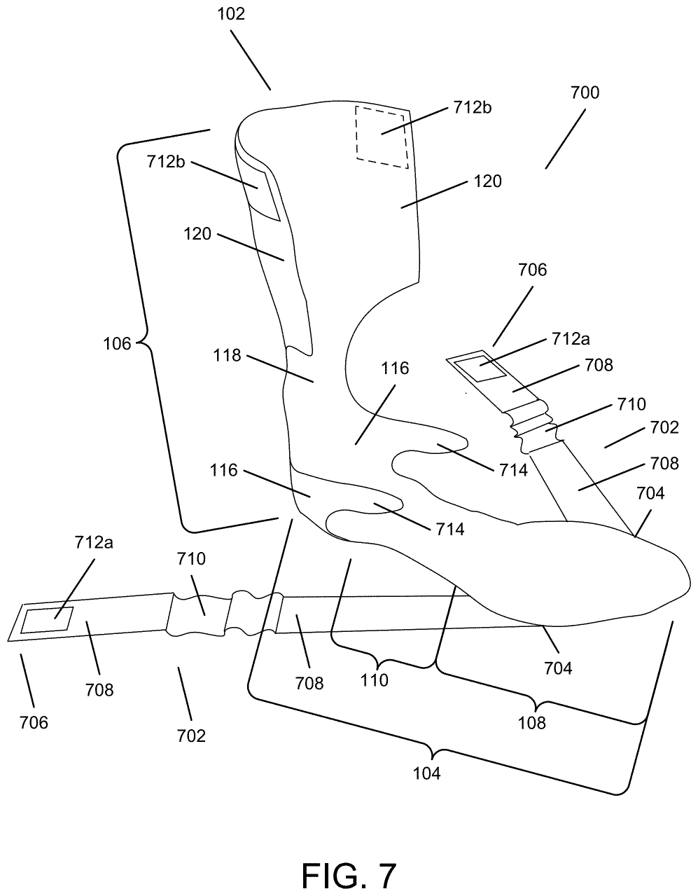

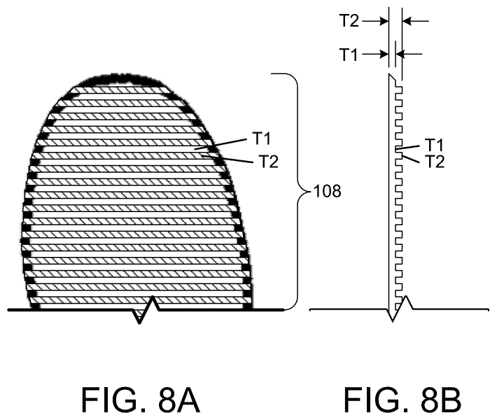

FIG. 8A illustrates, in simplified form, an underside 802 of a phalangeal portion 108 having such a configuration with alternating thicknesses T1 and T2, where T1 is between about 0.8 mm and 1 mm, and T2 is between about 1.5 mm and 2 mm. FIG. 8B illustrates, in simplified form, a side view of the underside 802 of FIG. 8A. The differential thickness can be produced additively, if made using additive manufacturing (aka 3D printing) or by adding discrete ribs, or subtractively, by starting with a thicker section and removing material to thin certain areas. Alternatively, the differential thickness can be made by compressing a thicker piece of material in discrete locations so as to achieve the desired thickness in those areas (although the compression approach may need to be followed by some manner of trimming of excess material.

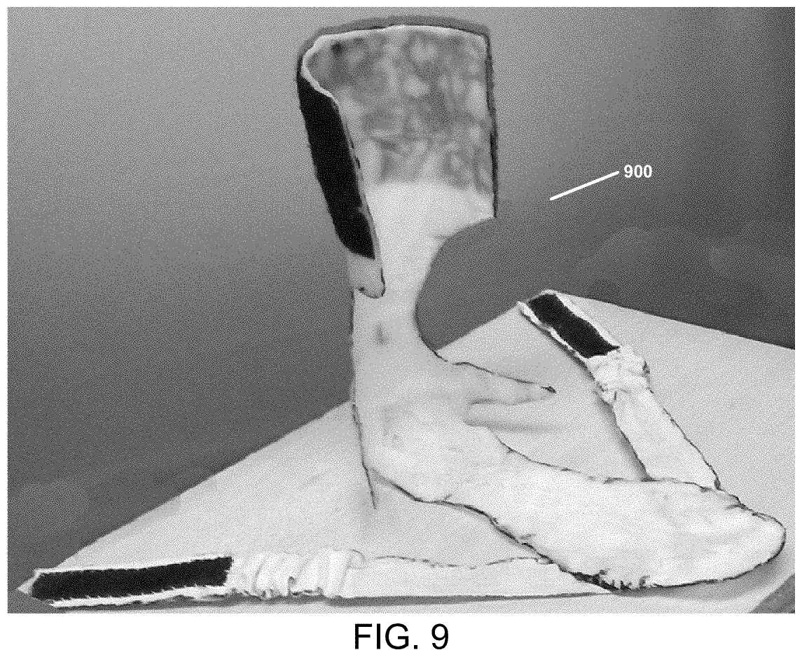

FIG. 9 is a photograph of an example AFO 900 that is essentially an implementation of the AFO 700 of FIG. 7.

Manufacture of the AFO

In accordance with the above, AFOs made according to the teachings herein are formed from a thermoplastic, typically of a polymers of the polyurethane/polyester type, and ideally, are made of polycaprolactone. However, other plastics can be used provided that they can provide the locational flexibility and rigidity described herein at a nominal thickness of between 0.8 mm and 2.5 mm. At this point, it should be noted that, as used herein, the term “nominal thickness” is intended to address the fact that the molding or printing process that will typically be used to form an AFO as described herein, can result in localized variations in thickness at individual locations of slightly less or more than the thickness specified. Thus, an areas specified as having a “nominal thickness” of 1 mm could actually have individual locations where the thickness could vary, for example, as 1 mm plus or minus 0.5 mm. However, the majority of the specified area should have an average thickness within that range.

An advantage to the use of polycaprolactone is that it softens at about 60 degrees Celsius (about 140 degrees Fahrenheit) when at least 2 mm thick, but is quite rigid at room temperature (i.e., about 20-21 degrees Celsius/about 68-78 degrees Fahrenheit) when at least 2 mm thick. And it is sufficiently flexible at room temperature when uniformly between 0.8 mm and 1.5 mm thick. This makes it easy to custom mold it to a given user. Thus, if minor adjustments are required, they can be performed by heating the area that needs to be adjusted, to about 60 degrees Celsius, which is not so hot as would prevent manipulation by hand.

Alternatively, AFOs constructed in accordance with the teachings herein can be constructed using 3D printing, using the aforementioned polycaprolactone or such other plastics as may be available at the time, for example, filaments of: polylactic acid (PLA), polyethylene terephthalate glycol (PETG), thermoplastic polyurethane (TPU), polypropylene (PP), or some combination thereof in 3D printing the AFO. The 3D printing method of manufacture allows for the ability to use one of the foregoing plastics or different plastics in different areas of the same AFO (if desired) to provide highly specific control of the flexibility/rigidity/thickness in individual areas, conformance to the user, and/or overall shape. The only limitation is the ability of the two different plastics to bond together or be bonded to each other. By way of example, the area of the AFO that would be behind the lower back part of the fibula and tibia can be 3D printed with a plastic that is rigid and strong, whereas an area of the AFO that needs to be very flexible can be 3D printed using a different plastic that has the desired flexibility/thickness combination. Thus, for example, if it is desired that the phalangeal section, heel section and any section therebetween have a uniform thickness, the heel section can be 3D printed with a plastic that is substantially rigid at the specified thickness (in that it appropriately limits plantar flexion, while potentially inhibiting or allowing flex in some other direction(s)), whereas the phalangeal section can be 3D printed using a different plastic that has the desired flexibility at that same specified thickness. Optionally, the section therebetween, if any, can be formed from the same plastic as either the phalangeal or heel sections, or can be formed at the specified thickness from yet a different plastic. The ability to form different portions of an AFO using different plastics allows for greater customizability than could have previously been achieved.

Moreover, once formed by 3D printing, if minor adjustments are required for the user, they can be performed by heating the area that needs to be adjusted to just above the glass transition temperature of the specific material in that area.

The creation process would be performed by scanning the user's foot, using, for example, a 3D scanner, and, possibly a flatbed scanner for the user's sole. The resulting scan of the users outer dimensions will then be used to form the inner dimensions of the AFO and the necessary thicknesses of the AFO will then be applied or specified in a direction outwards (i.e., in a direction away from the user's foot) from there, for example, using a pre-specified generic template, with the result being an .STL or other format file that can be supplied to a 3D printer to actually form the AFO. As to the 3D printing itself any 3D printer that is capable of printing the specified material at the desired thicknesses can be used.

Using the AFOs

As briefly noted above, an advantage of some of the AFOs as described herein is the ease of use. Specifically, given the flexible and rigid parts, a user of an AFO as described herein or constructed according to the teachings herein will initially insert the AFO into the user's footwear and then the user will insert their foot into the AFO footwear combination and then, if appropriate, tie, buckle, or otherwise tighten the footwear in the normal manner. In this way, virtually any of the user's footwear (e.g., sneakers, sandals, dress shoes, boots, and sandals of a type that do not require a piece to go between two of the user's toes (e.g., the hallux toe and index toe)). This is in sharp contrast to conventional AFOs which are affixed to the users foot and then the foot/conventional AFO combination must be inserted into the footwear, which is difficult and/or limits the footwear that the user can wear with conventional AFOs, due to the difficulty in insertion of the foot/conventional AFO combination.

Moreover, for cases where the need for the AFO is based upon weakness in the foot muscles, AFOs constructed according to the teachings herein that allow for plantar flexion of the at least part of the metatarsal section 110 can use flexible straps that provide enough resistance to plantar flexion to allow the user to exercise those muscles, while otherwise preventing undesirable plantar flexion during walking. Moreover, as the muscle strength improves, higher resistance straps can be sequentially substituted.

Having described and illustrated the principles of this application by reference to one or more examples, it should be apparent that embodiment(s) may be constructed and/or modified in arrangement and detail without departing from the principles disclosed herein and that it is intended that the application be construed as including all such modifications and variations insofar as they come within the spirit and scope of the subject matter disclosed.

The foregoing outlines, generally, the features and technical advantages of one or more implementations that can be constructed based upon the teachings in this disclosure in order that the following detailed description may be better understood. However, the advantages and features described herein are only a few of the many advantages and features available from representative examples of possible variant implementations and are presented only to assist in understanding. It should be understood that they are not to be considered limitations on the invention as defined by the appended claims, or limitations on equivalents to the claims. For instance, some of the advantages or aspects of different variants are mutually contradictory, in that they cannot be simultaneously present in a single embodiment. Similarly, some features or advantages may be applicable to one aspect and inapplicable to others. Thus, the foregoing features and advantages should not be considered dispositive in determining equivalence. Additional features and advantages will be apparent from the teachings of the description, drawings, and claims.

What is claimed is:

1. An ankle foot orthosis (AFO) for use on the lower limb of a person comprising:
a thermoplastic shell, made exclusively of a single specific thermoplastic, the thermoplastic shell comprising
a phalangeal section dimensioned to underlay the phalanges bones when worn by the person on their lower limb;
a heel section dimensioned to at least underlay the cuneiform bones and calcaneus when worn by the person on their lower limb; and
an upright portion coupled to a proximal portion of the heel section dimensioned to extend, substantially perpendicular to the heel section, from the heel section to just below the gastrocnemius muscle, while, when worn by the person on their lower limb, covering both the calcaneal tendon and at least a portion of the posterior soleus, and not covering the ankle joint;
wherein a portion of the shell, extending from a phalangeal side of the heel section through the phalangeal section, is flexible in a plantar flexion direction; and
wherein the upright portion is substantially rigid so as to inhibit plantar flexion of the person's foot when worn by the person.

2. The ankle foot orthosis of claim 1, wherein the thermoplastic shell comprises polycaprolactone.

3. The ankle foot orthosis of claim 1, wherein at least a portion of the thermoplastic shell is molded.

4. The ankle foot orthosis of claim 1, wherein at least a portion of the thermoplastic shell is 3d printed.

5. The ankle foot orthosis of claim 1, further comprising a metatarsal section, between the phalangeal section and heel section, the metatarsal section dimensioned to underlay at least a portion of the person's metatarsal bones.

6. The ankle foot orthosis of claim 5, wherein at least a distal portion of the metatarsal section is flexible.

7. The ankle foot orthosis of claim 1, further comprising a strap coupled to the phalangeal section.

8. The ankle foot orthosis of claim 7, wherein the strap includes an elastic section and a non-elastic section.

9. The ankle foot orthosis of claim 1, further comprising a pair of extensions extending in a longitudinal direction away from the heel section.

10. The ankle foot orthosis of claim 1, wherein the thermoplastic shell comprises a plastic that is soft and moldable at a temperature of about 60 degrees Celsius or more and rigid at a room temperature of between 20 and 21 degrees Celsius.

11. An ankle foot orthosis (AFO) for use on the lower limb of a person comprising:
an exclusively polycaprolactone shell, including
a phalangeal section dimensioned to underlay the phalanges bones when worn by the person on their lower limb;
a heel section dimensioned to at least underlay the cuneiform bones and calcaneus when worn by the person on their lower limb; and
an upright portion coupled to a proximal portion of the heel section dimensioned to extend, substantially perpendicular to the heel section, from the heel section to just below the gastrocnemius muscle, while, when worn by the person on their lower limb, covering both the calcaneal tendon and at least a portion of the posterior soleus, and not covering the ankle joint;
wherein a portion of the exclusively polycaprolactone shell, extending from a phalangeal side of the heel section through the phalangeal section is flexible in a plantar flexion direction; and
wherein the upright portion is substantially rigid so as to inhibit plantar flexion of the person's foot.

12. The ankle foot orthosis of claim 11, wherein the exclusively polycaprolactone shell is a molded single sheet of polycaprolactone.

13. A 3D printed ankle foot orthosis (AFO) for use on the lower limb of a person comprising:
a unitary shell including multiple layers of exclusively a single plastic, the shell further having
a phalangeal section of dimensioned to underlay the phalanges bones when worn by the person on their lower limb;
a heel section dimensioned to at least underlay the cuneiform bones and calcaneus when worn by the person on their lower limb; and
an upright portion coupled to a proximal portion of the heel section dimensioned to extend, substantially perpendicular to the heel section, from the heel section to just below the gastrocnemius muscle, while, when worn by the person on their lower limb, covering both the calcaneal tendon and at least a portion of the posterior soleus, and not covering the ankle joint;
wherein a portion of the exclusively polycaprolactone shell, extending from a phalangeal side of the heel section through the phalangeal section is flexible in a plantar flexion direction; and
wherein the upright portion is substantially rigid so as to inhibit plantar flexion of the person's foot.

14-16. (canceled)

17. The 3D printed ankle foot orthosis of claim 13, wherein the single plastic is one of:
polylactic acid (PLA), polyethylene terephthalate glycol (PETG), thermoplastic polyurethane (TPU), or polypropylene (PP).

18-19. (canceled)

20. The 3D printed ankle foot orthosis of claim 13, wherein the phalangeal section and the heel section have a common thickness.
